(19) United States
(12) Patent Application Publication
(10) Pub. No.: US 2022/0241165 A1
(43) Pub. Date: Aug. 4, 2022

(54) ORAL CARE COMPOSITIONS COMPRISING OXALIC ACID

(71) Applicant: The Procter & Gamble Company, Cincinnati, OH (US)

(72) Inventors: Phillip Asa Drake, Mason, OH (US); Andrew Frederic Groth, Mason, OH (US); Tiffany Celeste Hare, West Chester, OH (US); Paul Albert Sagel, Maineville, OH (US); Rachel Anne Trenner, Centerville, OH (US)

(21) Appl. No.: 17/580,685

(22) Filed: Jan. 21, 2022

Related U.S. Application Data
(60) Provisional application No. 63/143,968, filed on Feb. 1, 2021.

(51) Int. Cl. A61K 8/362; A61K 8/21; A61K 8/27; A61K 8/19; A61K 8/24; A61K 8/25; A61Q 11/00

(52) U.S. Cl. CPC A61K 8/362; A61K 8/21; A61K 8/27; A61K 8/19; A61K 2800/28; A61K 8/25; A61Q 11/00; A61K 2800/48; A61K 8/24

(57) ABSTRACT

Oral care compositions comprising oxalic acid and pH buffering agent with a pKₐ of from about 4 to about 6.5. Oral care compositions comprising oxalic acid and a pH buffering agent, such as adipic acid, glutaric acid, succinic acid, malonic acid, glutamic acid, ascorbic acid, citric acid, salts thereof, or combinations thereof.

### FIELD OF THE INVENTION

The present invention relates to oral care compositions comprising oxalic acid and a pH buffering agent. The present invention relates to oral care compositions comprising oxalic acid and a pH buffering agent with a pKₐ of from about 4 to about 6.5. The present invention also relates to oral care compositions comprising oxalic acid that provide a sensitivity benefit.

### BACKGROUND OF THE INVENTION

The four major components of human teeth are enamel, cementum, pulp, and dentin. The dentin layer of teeth includes microscopic channels, known as dentinal tubules, which run from the pulp to the exterior cementum layer (below the gum line) or the exterior enamel layer (above the gum line).

The dentin layer, which is naturally protected and sealed by enamel or cementum, can become exposed through gum recession, enamel wear, and/or erosion. Once the dentin layer is exposed, variations in temperature, tactile sensations, and/or osmotic insults can cause a rapid pulse of fluid through the exposed dentin tubules. The rapid pulse of fluid stimulates the pulpal nerve and leads to transient pain in the oral cavity, which is commonly referred to as tooth sensitivity.

Tooth sensitivity can be treated by forming a barrier over the exposed dentin and/or filling the dentin tubules with a solid material. Stannous ions can be added to oral care compositions to provide a tooth sensitivity relief by facilitating the formation of a physical barrier. However, the sensitivity benefit can be temporary as the physical barrier may not be long lasting.

Longer lasting tooth sensitivity can be achieved by precipitating and/or crystallizing solid material within the dentin tubules. Crystallization of material into dentin tubules can be preferred because crystalized material can be more resistant to subsequent solubilization after deposition within the dentin tubules. However, achieving the crystallization of material into the dentin tubules can be challenging to accomplish in a short period of time suitable for a single oral health session, such as brushing teeth or using a mouthwash.

Thus, there is a need for oral care compositions that can effectively and quickly crystallize material into dentin tubules.

### SUMMARY OF THE INVENTION

Disclosed herein are oral care compositions that can crystalize material into dentin tubules. The disclosed oral care compositions can lead to crystalized material in exposed dentin tubules during a single oral care regimen.

Disclosed herein is an oral care composition comprising (a) oxalic acid; and (b) a pH buffering agent with a pKₐ of from about 4 to about 6.5.

Also disclosed herein are methods of use of the disclosed oral care compositions to prevent, treat, and/or mitigate tooth and/or gum sensitivity in the oral cavity.

### BRIEF DESCRIPTION OF THE DRAWINGS

FIG. 1 shows the schematic of a liquid cell, exploded (left) and assembled cell (right), which was utilized to mount dentin disk samples for conditioning and application of antisensitivity test solutions. When incorporated in the apparatus shown in FIG. 2, the cell also enabled simulation of the in vivo treatment environment in that (1) outward pulpal flow is maintained during treatment application, and (2) the accessible surface of the dentin permits direct treatment application as would occur in mouth.

FIG. 2 shows the apparatus used to supply liquid under pressure to the cell described in FIG. 1. Note that the apparatus was configured to separately exert two distinct biologically-relevant pressure differentials across the dentin section. The modest pressure exerted by the height difference between the cell and liquid level in the reservoir is meant to be commensurate with normal in vivo pulpal pressure. Conversely, the 5 psi (34.5 kPa) pressure applied via the regulator is intended to approximate a pressure differential associated with moderate in vivo sensitivity pain.

FIG. 3 shows a plot of pH vs volumetric flow reduction in human dentin using the in vitro Hydrodynamic Flow (HF) model before and after a single application of approximately 200 μL of an aqueous 1.5% solution of oxalate ion to the dentin surface for 5 seconds.

FIG. 4 shows a plot of pH vs volumetric flow reduction (performance) using the HF model in human dentin before and after a single application of 200 μL of an aqueous solution of 1.5% oxalate ion for 10 minutes. The curved line was obtained with a non-linear curve fit to a simple exponential decay profile. Note the differences in pH range studied in FIG. 3 and FIG. 4.

FIG. 5 shows a plot of in situ pH change before and after application of unbuffered pH 5.2 oxalate solution using the Salivary Dilution and Neutralization (SDN) model. Note the reduction in salivary pH immediately after product application, after which the pH rapidly increases, presumably as a result of interaction with soft tissue and salivary stimulation.

FIG. 6 shows a plot of in situ pH change before and after application of an unbuffered oxalate control versus a buffered oxalate solution, where both solutions were adjusted to pH 4.2 before application.

FIG. 7 shows a plot of in situ pH change before and after application of an unbuffered oxalate control versus a buffered oxalate solution, where both solutions were adjusted to pH 5.2 before application.

FIG. 8 shows a plot of in situ pH change before and after application of an unbuffered oxalate control versus a buffered oxalate solution, where both solutions were adjusted to pH 7.0 before application.

FIG. 9 shows a plot of in vitro efficacy measured in the HF model for an unbuffered oxalate control versus a buffered oxalate solution, both mixed with whole, stimulated saliva at a ratio and pH to mimic in situ conditions. Control and treatment solutions were formulated at pH 4.2.

FIG. 10 shows a plot of in vitro efficacy measured in the HF model for an unbuffered oxalate control versus a buffered oxalate solution, both mixed with whole, stimulated saliva at a ratio and pH to mimic in situ conditions. Control and treatment solutions were formulated at pH 5.2.

FIG. 11 shows a plot of in vitro efficacy measured in the HF model for an unbuffered oxalate control versus a buffered oxalate solution, both mixed with whole, stimulated saliva at a ratio and pH to mimic in situ conditions. Control and treatment solutions were formulated at pH 7.0.

### DETAILED DESCRIPTION OF THE INVENTION

The present invention is directed to oral care compositions that can crystalize material into dentin tubules to reduce tooth sensitivity in the oral cavity of an affected individual. Oxalic acid, and salts thereof, is known to provide sensitivity benefit through partial or full occlusion of dentin tubules through the precipitation and/or crystallization of oxalate salts, such as calcium oxalate.

Without wishing to be bound by theory, it is believed that the amount and rate of crystallization of oxalate salts into dentin tubules is impacted by the concentration of oxalic acid/oxalate, time, and pH during treatment. Without wishing to be bound by theory, it has been found that maintaining a certain pH can lead to increased rates of crystallization of oxalate salts into dentin tubules. Thus, the disclosed oral care compositions include oxalic acid, or salts thereof, and a pH buffering agent with a pKₐ of from about 4 to about 6.5. Alternatively, the disclosed oral care compositions include oxalic acid, or salts thereof, and a pH buffering agent capable of buffering a solution to maintain a pH of from about 4 to about 6.5 during the treatment event.

### Definitions

To define more clearly the terms used herein, the following definitions are provided. Unless otherwise indicated, the following definitions are applicable to this disclosure. If a term is used in this disclosure but is not specifically defined herein, the definition from the IUPAC Compendium of Chemical Terminology, 2nd Ed (1997), can be applied, as long as that definition does not conflict with any other disclosure or definition applied herein, or render indefinite or non-enabled any claim to which that definition is applied.

The term “oral care composition”, as used herein, includes a product, which in the ordinary course of usage, is not intentionally swallowed for purposes of systemic administration of particular therapeutic agents, but is rather retained in the oral cavity for a time sufficient to contact dental surfaces or oral tissues. Examples of oral care compositions include dentifrice, tooth gel, subgingival gel, mouth rinse, mousse, foam, mouth spray, lozenge, chewable tablet, chewing gum, tooth whitening strips, floss and floss coatings, breath freshening dissolvable strips, or denture care or adhesive product. The oral care composition may also be incorporated onto strips or films for direct application or attachment to oral surfaces.

The term “dentifrice composition”, as used herein, includes tooth or subgingival -paste, gel, or liquid formulations unless otherwise specified. The dentifrice composition may be a single-phase composition or may be a combination of two or more separate dentifrice compositions. The dentifrice composition may be in any desired form, such as deep striped, surface striped, multilayered, having a gel surrounding a paste, or any combination thereof. Each dentifrice composition in a dentifrice comprising two or more separate dentifrice compositions may be contained in a physically separated compartment of a dispenser and dispensed side-by-side.

“Active and other ingredients” usefpL herein may be categorized or described herein by their cosmetic and/or therapeutic benefit or their postulated mode of action or function. However, it is to be understood that the active and other ingredients usefpL herein can, in some instances, provide more than one cosmetic and/or therapeutic benefit or function or operate via more than one mode of action. Therefore, classifications herein are made for the sake of convenience and are not intended to limit an ingredient to the particularly stated function(s) or activities listed.

The term “orally acceptable carrier” comprises one or more compatible solid or liquid excipients or diluents which are suitable for topical oral administration. By “compatible,” as used herein, is meant that the components of the composition are capable of being commingled without interaction in a manner which would substantially reduce the composition's stability and/or efficacy. The carriers or excipients of the present invention can include the usual and conventional components of mouthwashes or mouth rinses, as more fully described hereinafter: Mouthwash or mouth rinse carrier materials typically include, but are not limited to one or more of water, alcohol, humectants, surfactants, and acceptance improving agents, such as flavoring, sweetening, coloring and/or cooling agents.

The term “substantially free” as used herein refers to the presence of no more than 0.05%, preferably no more than 0.01%, and more preferably no more than 0.001%, of an indicated material in a composition, by total weight of such composition.

The term “essentially free” as used herein means that the indicated material is not deliberately added to the composition, or preferably not present at analytically detectable levels. It is meant to include compositions whereby the indicated material is present only as an impurity of one of the other materials deliberately added.

The term “oral hygiene regimen” or “regimen” can be for the use of two or more separate and distinct treatment steps for oral health. e.g. toothpaste, mouth rinse, floss, toothpicks, spray, water irrigator, massager.

The term “total water content” as used herein means both free water and water that is bound by other ingredients in the oral care composition.

For the purpose of the present invention, the relevant molecular weight (MW) to be used is that of the material added when preparing the composition e.g., if the chelant is a citrate species, which can be supplied as citric acid, sodium citrate or indeed other salt forms, the MW used is that of the particular salt or acid added to the composition but ignoring any water of crystallization that may be present.

While compositions and methods are described herein in terms of “comprising” various components or steps, the compositions and methods can also “consist essentially of” or “consist of” the various components or steps, unless stated otherwise.

As used herein, the word “or” when used as a connector of two or more elements is meant to include the elements individually and in combination; for example, X or Y, means X or Y or both.

As used herein, the articles “a” and “an” are understood to mean one or more of the material that is claimed or described, for example, “an oral care composition” or “a bleaching agent.”

All measurements referred to herein are made at about 23° C. (i.e. room temperature) unless otherwise specified.

Generally, groups of elements are indicated using the numbering scheme indicated in the version of the periodic table of elements published in Chemical and Engineering News, 63(5), 27, 1985. In some instances, a group of elements can be indicated using a common name assigned to the group; for example, alkali metals for Group 1 elements, alkaline earth metals for Group 2 elements, and so forth.

Several types of ranges are disclosed in the present invention. When a range of any type is disclosed or claimed, the intent is to disclose or claim individually each possible number that such a range could reasonably encompass, including end points of the range as well as any sub-ranges and combinations of sub-ranges encompassed therein.

The term “about” means that amounts, sizes, formulations, parameters, and other quantities and characteristics are not and need not be exact, but can be approximate and/or larger or smaller, as desired, reflecting tolerances, conversion factors, rounding off, measurement errors, and the like, and other factors known to those of skill in the art. In general, an amount, size, formulation, parameter or other quantity or characteristic is “about” or “approximate” whether or not expressly stated to be such. The term “about” also encompasses amounts that differ due to different equilibrium conditions for a composition resulting from a particular initial mixture. Whether or not modified by the term “about,” the claims include equivalents to the quantities. The term “about” can mean within 10% of the reported numerical value, preferably within 5% of the reported numerical value.

The dentifrice composition can be in any suitable form, such as a solid, liquid, powder, paste, or combinations thereof. The oral care composition can be dentifrice, tooth gel, subgingival gel, mouth rinse, mousse, foam, mouth spray, lozenge, chewable tablet, chewing gum, tooth whitening strips, floss and floss coatings, breath freshening dissolvable strips, or denture care or adhesive product. The components of the dentifrice composition can be incorporated into a film, a strip, a foam, or a fiber-based dentifrice composition.

The oral care compositions, as described herein, comprise oxalic acid and pH buffering agent. Additionally, the oral care compositions can comprise other optional ingredients, as described below. The section headers below are provided for convenience only. In some cases, a compound can fall within one or more sections. For example, stannous fluoride can be a tin compound and/or a fluoride compound.

### Oxalic Acid

The oral care composition comprises oxalic acid. The oxalic acid can comprise suitable salts of oxalic acid, such as, for example, monoalkali metal oxalate, dialkali metal oxalate, monopotassium monohydrogen oxalate, dipotassium oxalate, monosodium monohydrogen oxalate, disodium oxalate, titanium oxalate, and/or other metal salts of oxalate. The oxalic acid can also include hydrates of the oxalic acid and/or a hydrate of a salt of the oxalic acid.

The oral care composition can comprise from about 0.01% to about 10%, from about 0.1% to about 15%, from about 0.1% to about 5%, or from about 0.0001 to about 25%, of oxalic acid.

pH buffering agent

The oral care composition comprises pH buffering agent, which can adjust the pH, but also provide buffering capacity. The pH buffering agent can have a pKₐ of from about 4 to about 6.5, from about 4.5 to about 6, from about 5 to about 6, or from about 4 to about 7.

The pH buffering agent can comprise monocarboxylic acid, dicarboxylic acid, tricarboxylic acid, tetracarboxylic acid, or combinations thereof.

The monocarboxylic acid can comprise a single carboxylic acid functional group. Suitable compounds can include compounds with the formula R—COOH, wherein R is any organic structure. Suitable monocarboxylic acids can also include aliphatic carboxylic acid, aromatic carboxylic acid, sugar acid, salts thereof, and/or combinations thereof.

The aliphatic carboxylic acid can comprise a carboxylic acid functional group attached to a linear hydrocarbon chain, a branched hydrocarbon chain, and/or cyclic hydrocarbon molecule. The aliphatic carboxylic acid can be fully saturated or unsaturated and have one or more alkene and/or alkyne functional groups. Other functional groups can be present and bonded to the hydrocarbon chain, including halogenated variants of the hydrocarbon chain. The aliphatic carboxylic acid can also include hydroxyl acids, which are organic compounds with an alcohol functional group in the alpha, beta, or gamma position relative to the carboxylic acid functional group. A suitable alpha hydroxy acid includes lactic acid and/or a salt thereof.

The aromatic carboxylic acid can comprise a carboxylic acid functional group attached to at least one aromatic functional group. Suitable aromatic carboxylic acid groups can include benzoic acid, salicylic acid, and/or combinations thereof.

The carboxylic acid can include formic acid, acetic acid, propionic acid, butyric acid, valeric acid, caproic acid, enanthic acid, caprylic acid, ascorbic acid, benzoic acid, caprylic acid, cholic acid, glycine, alanine, valine, isoleucine, leucine, phenylalanine, linoleic acid, niacin, oleic acid, propanoic acid, sorbic acid, stearic acid, gluconate, lactate, carbonate, chloroacetic and/or combinations thereof.

The pH buffering agent can comprise dicarboxylic acid. The dicarboxylic acid comprises a compound with two carboxylic acid functional groups. The dicarboxylic acid can comprise a compound or salt thereof defined by Formula I-A, Formula I-B, and/or Formula I-C.

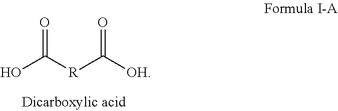

R can be alkyl, alkenyl, allyl, phenyl, benzyl, acetyl, aliphatic, aromatic, polyethylene glycol, polymer, O, N, P, or combinations thereof. R can also be additionally functionalized with one or more functional groups, such as —OH, —NH₂, and/or alkyl, alkenyl, aromatic, or combinations thereof.

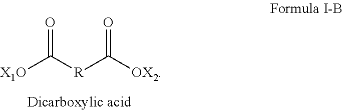

R can be alkyl, alkenyl, allyl, phenyl, benzyl, acetyl, aliphatic, aromatic, polyethylene glycol, polymer, O, N, P, or combinations thereof. R can also be additionally functionalized with one or more functional groups, such as —OH, —NH₂, and/or alkyl, alkenyl, aromatic, or combinations thereof.

X₁ and X₂ can independently be H, alkali metal, alkali earth metal, transition metal, or combinations thereof. Suitable alkali metals include lithium, sodium, potassium, or combinations thereof. Suitable alkali earth metals include magnesium, calcium, barium, or combinations thereof. Suitable transitional metals include titanium, chromium, iron, nickel, copper, zinc, tin, gold, silver, or combinations thereof.

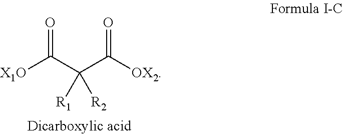

R₁ can be alkyl, alkenyl, allyl, phenyl, benzyl, acetyl, aliphatic, aromatic, polyethylene glycol, polymer, O, N, P, or combinations thereof. R can also be additionally functionalized with one or more functional groups, such as —OH, —NH₂, and/or alkyl, alkenyl, aromatic, or combinations thereof.

X₁ and X₂ can independently be H, alkali metal, alkali earth metal, transition metal, or combinations thereof. Suitable alkali metals include lithium, sodium, potassium, or combinations thereof. Suitable alkali earth metals include magnesium, calcium, barium, or combinations thereof. Suitable transitional metals include titanium, chromium, iron, nickel, copper, zinc, tin, gold, silver, or combinations thereof.

The dicarboxylic acid can be added to a formulation as a neutral acid (as shown in Formula I-A) or as a dicarboxylate monosalt (where one of the carboxylic acid functional groups is a salt and the other is neutral), a dicarboxylate disalt (where both of the carboxylic acid functional groups are salts), or combinations thereof. Additionally, as is well known to a person of ordinary skill in the art, whether or not that one or both of the carboxylic acid functional groups of the dicarboxylic acid are neutral or charged in solution, can be influenced by the pH of the solution. For example, a neutral dicarboxylic acid can be added to an aqueous solution and one or two protons from the two carboxylic acid functional groups can be removed if the pH is lower than the pKa of the carboxylic acid functional group, as shown below in Formula I-D.

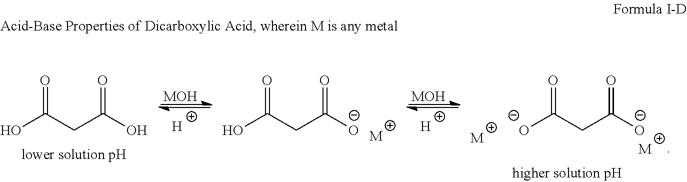

The dicarboxylic acid can comprise malonic acid, succinic acid, glutaric acid, adipic acid, pimelic acid, suberic acid, azerlaic acid, sebacic acid, undecanedioic acid, dodecanedioic acid, brassylic acid, thapsic acid, japanic acid, phellogenic acid, equisetolic acid, malic acid, maleic acid, tartaric acid, phthalic acid, methylmalonic acid, dimethylmalonic acid, tartronic acid, mesoxalic acid, dihydroxymalonic acid, dihydroxymalonic acid, fumaric acid, terephthalic acid, glutaric acid, salts thereof, or combinations thereof. The dicarboxylic acid can comprise suitable salts of dicarboxylic acid, such as, for example, when the dicarboxylic acid includes a salt of oxalic acid: monoalkali metal oxalate, dialkali metal oxalate, monopotassium monohydrogen oxalate, dipotassium oxalate, monosodium monohydrogen oxalate, disodium oxalate, titanium oxalate, and/or other metal salts of oxalate. The dicarboxylic acid can also include hydrates of the dicarboxylic acid and/or a hydrate of a salt of the dicarboxylic acid. Suitable dicarboxylic acid compounds include dicarboxylic acids described by Formula I-A, wherein R is null, comprises a methylene or ethylene with one or two substitutions, and/or an acetyl group.

Other suitable pH buffering agents include adipic acid, glutaric acid, succinic acid, malonic acid, glutamic acid, ascorbic acid, citric acid, and/or combinations thereof.

The oral care composition can comprise from about 0.01% to about 10%, from about 0.1% to about 15%, from about 1% to about 5%, or from about 0.0001 to about 25%, of pH buffering agent.

pH

The pH of the oral care compositions as described herein can be from about 4 to about 7, from about 4 to about 6.5, from about 4.5 to about 6, from about 4.5 to about 5.5, or from about 4 to about 5.5. The pH of a mouthrinse solution can be determined as the pH of the neat solution. The pH of a dentifrice composition can be determined as a slurry pH, which is the pH of a mixture of the dentifrice composition and water, such as a 1:4, 1:3, or 1:2 mixture of the dentifrice composition and water.

The pH of the oral care compositions as described herein have a preferred pH of below about 7 or below about 6 due to the pKa of oxalic acid and the pH buffering agent. While not wishing to be bound by theory, it is believed that oxalic acid displays unique behavior when the pH is below about 7 or below about 6, but surfaces in the oral cavity can also be sensitive to a low pH. Additionally, at pH values above about pH 7, the metal ion source can react with water and/or hydroxide ions to form insoluble metal oxides and/or metal hydroxides. The formation of these insoluble compounds can limit the ability of dicarboxylates to stabilize metal ions in oral care compositions and/or can limit the interaction of dicarboxylates with target metal ions in the oral cavity.

Additionally, at pH values less than 4, the potential to damage teeth by acid dissolution is greatly increased. Consequently, the oral care compositions comprising oxalic acid, as described herein, preferably have a pH from about 4 to about 7, from about 4 to about 6, from about 4.5 to about 6.5, from about 4.5 to about 5.5, or from about 4 to about 5.5 to minimize metal hydroxide/metal oxide formation and any damage to oral hard tissues (enamel, dentin, and cementum).

### Fluoride

The oral care composition can comprise fluoride, which can be provided by a fluoride ion source. The fluoride ion source can comprise one or more fluoride containing compounds, such as stannous fluoride, sodium fluoride, potassium fluoride, amine fluoride, sodium monofluorophosphate, zinc fluoride, and/or mixtures thereof.

The fluoride ion source and the tin ion source can be the same compound, such as for example, stannous fluoride, which can generate tin ions and fluoride ions. Additionally, the fluoride ion source and the tin ion source can be separate compounds, such as when the tin ion source is stannous chloride and the fluoride ion source is sodium monofluorophosphate or sodium fluoride.

The fluoride ion source and the zinc ion source can be the same compound, such as for example, zinc fluoride, which can generate zinc ions and fluoride ions. Additionally, the fluoride ion source and the zinc ion source can be separate compounds, such as when the zinc ion source is zinc phosphate and the fluoride ion source is stannous fluoride.

The fluoride ion source can be essentially free of, or free of stannous fluoride. Thus, the oral care composition can comprise sodium fluoride, potassium fluoride, amine fluoride, sodium monofluorophosphate, zinc fluoride, and/or mixtures thereof.

The oral care composition can comprise a fluoride ion source capable of providing from about 50 ppm to about 5000 ppm, and preferably from about 500 ppm to about 3000 ppm of free fluoride ions. To deliver the desired amount of fluoride ions, the fluoride ion source may be present in the oral care composition at an amount of from about 0.0025% to about 5%, from about 0.01% to about 10%, from about 0.2% to about 1%, from about 0.5% to about 1.5%, or from about 0.3% to about 0.6%, by weight of the oral care composition. Alternatively, the oral care composition can comprise less than 0.1%, less than 0.01%, be essentially free of, be substantially free of, or free of a fluoride ion source.

### Metal

The oral care composition, as described herein, can comprise metal, which can be provided by a metal ion source comprising one or more metal ions. The metal ion source can comprise or be in addition to the tin ion source and/or the zinc ion source, as described herein. Suitable metal ion sources include compounds with metal ions, such as, but not limited to Sn, Zn, Cu, Mn, Mg, Sr, Ti, Fe, Mo, B, Ba, Ce, Al, In and/or mixtures thereof. The metal ion source can be any compound with a suitable metal and any accompanying ligands and/or anions.

Suitable ligands and/or anions that can be paired with metal ion sources include, but are not limited to acetate, ammonium sulfate, benzoate, bromide, borate, carbonate, chloride, citrate, gluconate, glycerophosphate, hydroxide, iodide, oxalate, oxide, propionate, D-lactate, DL-lactate, orthophosphate, pyrophosphate, sulfate, nitrate, tartrate, and/or mixtures thereof.

The oral care composition can comprise from about 0.01% to about 10%, from about 1% to about 5%, or from about 0.5% to about 15% of metal and/or a metal ion source.

### Tin

The oral care composition of the present invention can comprise tin, which can be provided by a tin ion source. The tin ion source can be any suitable compound that can provide tin ions in an oral care composition and/or deliver tin ions to the oral cavity when the oral care composition is applied to the oral cavity. The tin ion source can comprise one or more tin containing compounds, such as stannous fluoride, stannous chloride, stannous bromide, stannous iodide, stannous oxide, stannous oxalate, stannous sulfate, stannous sulfide, stannic fluoride, stannic chloride, stannic bromide, stannic iodide, stannic sulfide, and/or mixtures thereof. Tin ion source can comprise stannous fluoride, stannous chloride, and/or mixture thereof. The tin ion source can also be a fluoride-free tin ion source, such as stannous chloride.

The oral care composition can comprise from about 0.0025% to about 5%, from about 0.01% to about 10%, from about 0.2% to about 1%, from about 0.4% to about 1%, or from about 0.3% to about 0.6%, by weight of the oral care composition, of tin and/or a tin ion source. Alternatively, the oral care composition can be essentially free of, substantially free of, or free of tin.

### Zinc

The oral care composition can comprise zinc, which can be provided by a zinc ion source. The zinc ion source can comprise one or more zinc containing compounds, such as zinc fluoride, zinc lactate, zinc oxide, zinc phosphate, zinc chloride, zinc acetate, zinc hexafluorozirconate, zinc sulfate, zinc tartrate, zinc gluconate, zinc citrate, zinc malate, zinc glycinate, zinc pyrophosphate, zinc metaphosphate, zinc oxalate, and/or zinc carbonate. The zinc ion source can be a fluoride-free zinc ion source, such as zinc phosphate, zinc oxide, and/or zinc citrate.

The zinc and/or zinc ion source may be present in the total oral care composition at an amount of from about 0.01% to about 10%, from about 0.2% to about 1%, from about 0.4% to about 1%, or from about 0.3% to about 0.6%, by weight of the dentifrice composition. Alternatively, the oral care composition can be essentially free of, substantially free of, or free of zinc.

### Polyphosphate

The oral care composition can comprise polyphosphate, which can be provided by a polyphosphate source. A polyphosphate source can comprise one or more polyphosphate molecules. Polyphosphates are a class of materials obtained by the dehydration and condensation of orthophosphate to yield linear and cyclic polyphosphates of varying chain lengths. Thus, polyphosphate molecules are generally identified with an average number (n) of polyphosphate molecules, as described below. A polyphosphate is generally understood to consist of two or more phosphate molecules arranged primarily in a linear configuration, although some cyclic derivatives may be present.

Preferred polyphosphates are those having an average of two or more phosphate groups so that surface adsorption at effective concentrations produces sufficient non-bound phosphate functions, which enhance the anionic surface charge as well as hydrophilic character of the surfaces. Preferred thisin invention are the linear polyphosphates having the formula: XO(XPO₃)ₙX, wherein X is sodium, potassium, ammonium, or any other alkali metal cations and n averages from about 2 to about 21. Alkali earth metal cations, such as calcium, are not preferred because they tend to form insoluble fluoride salts from aqueous solutions comprising a fluoride ions and alkali earth metal cations. Thus, the oral care compositions disclosed herein can be free of or substantially free of calcium pyrophosphate.

Some examples of suitable polyphosphate molecules include, for example. pyrophosphate (n=2), tripolyphosphate (n=3), tetrapolyphosphate (n=4), sodaphos polyphosphate (n=6), hexaphos polyphosphate (n=13), benephos polyphosphate (n=14), hexametaphosphate (n=21), which is also known as Glass H. Polyphosphates can include those polyphosphate compounds manufactured by FMC Corporation, ICL Performance Products, and/or Astaris.

The oral care composition can comprise from about 0.01% to about 15%, from about 0.1% to about 10%, from about 0.5% to about 5%, from about 1 to about 20%, or about 10% or less, by weight of the oral care composition, of the polyphosphate source. Alternatively, the oral care composition can be essentially free of, substantially free of, or free of polyphosphate.

### Orthophosphate

The oral care composition can comprise orthophosphate, which can be provided by an orthophosphate source. An orthophosphate source can comprise a salt including the orthophosphate anion, a salt including a phosphate anion (H₂PO₄⁻, HPO₄²⁻, and PO₄³⁻), a phosphoric acid compound, a polyphosphate source, which can breakdown into orthophosphate under a variety of conditions, and/or another suitable orthophosphate source.

The oral care composition can comprise from about 0.01% to about 15%, from about 0.1% to about 10%, from about 0.5% to about 5%, from about 1 to about 20%, or about 10% or less, by weight of the oral care composition, of the orthophosphate. Alternatively, the oral care composition can be essentially free of, substantially free of, or free of orthophosphate.

### Surfactants

The oral care composition can comprise one or more surfactants. The surfactants can be used to make the compositions more cosmetically acceptable. The surfactant is preferably a detersive material which imparts to the composition detersive and foaming properties. Suitable surfactants are safe and effective amounts of anionic, cationic, nonionic, zwitterionic, amphoteric and betaine surfactants, such as sodium lauryl sulfate, sodium lauryl isethionate, sodium lauroyl methyl isethionate, sodium cocoyl glutamate, sodium dodecyl benzene sulfonate, alkali metal or ammonium salts of lauroyl sarcosinate, myristoyl sarcosinate, palmitoyl sarcosinate, stearoyl sarcosinate and oleoyl sarcosinate, polyoxyethylene sorbitan monostearate, isostearate and laurate, sodium lauryl sulfoacetate, N-lauroyl sarcosine, the sodium, potassium, and ethanolamine salts of N-lauroyl, N-myristoyl, or N-palmitoyl sarcosine, polyethylene oxide condensates of alkyl phenols, cocoamidopropyl betaine, lauramidopropyl betaine, palmityl betaine, sodium cocoyl glutamate, and the like. Sodium lauryl sulfate is a preferred surfactant. The oral care composition can comprise one or more surfactants each at a level from about 0.01% to about 15%, from about 0.3% to about 10%, or from about 0.3% to about 2.5%, by weight of the oral care composition.

### Thickening Agent

The oral care composition can comprise one or more thickening agents. Thickening agents can be usefpL in the oral care compositions to provide a gelatinous structure that stabilizes the toothpaste against phase separation. Suitable thickening agents include polysaccharides, polymers, and/or silica thickeners. Some non-limiting examples of polysaccharides include starch; glycerite of starch; gums such as gum karaya (sterculia gum), gum tragacanth, gum arabic, gum ghatti, gum acacia, xanthan gum, guar gum and cellulose gum; magnesium aluminum silicate (Veegum); carrageenan; sodium alginate; agar-agar; pectin; gelatin; cellulose compounds such as cellulose, carboxymethyl cellulose, hydroxyethyl cellulose, hydroxypropyl cellulose, hydroxymethyl cellulose, hydroxymethyl carboxypropyl cellulose, methyl cellulose, ethyl cellulose, and sulfated cellulose; natural and synthetic clays such as hectorite clays; and mixtures thereof.

The thickening agent can comprise polysaccharides. Polysaccharides that are suitable for use herein include carageenans, gellan gum, locust bean gum, xanthan gum, carbomers, poloxamers, modified cellulose, and mixtures thereof. Carageenan is a polysaccharide derived from seaweed. There are several types of carageenan that may be distinguished by their seaweed source and/or by their degree of and position of sulfation. The thickening agent can comprise kappa carageenans, modified kappa carageenans, iota carageenans, modified iota carageenans, lambda carrageenan, and mixtures thereof. Carageenans suitable for use herein include those commercially available from the FMC Company under the series designation “Viscarin,” including but not limited to Viscarin TP 329, Viscarin TP 388, and Viscarin TP 389.

The thickening agent can comprise one or more polymers. The polymer can be a polyethylene glycol (PEG), a polyvinylpyrrolidone (PVP), polyacrylic acid, a polymer derived from at least one acrylic acid monomer, a copolymer of maleic anhydride and methyl vinyl ether, a crosslinked polyacrylic acid polymer, of various weight percentages of the oral care composition as well as various ranges of average molecular ranges. The polymer can comprise polyacrylate crosspolymer, such as polyacrylate crosspolymer-6. Suitable sources of polyacrylate crosspolymer-6 can include Sepimax Zen™ commercially available from Seppic.

The thickening agent can comprise inorganic thickening agents. Some non-limiting examples of suitable inorganic thickening agents include colloidal magnesium aluminum silicate, silica thickeners. UsefμL silica thickeners include, for example, include, as a non-limiting example, an amorphous precipitated silica such as ZEODENT® 165 silica. Other non-limiting silica thickeners include ZEODENT® 153, 163, and 167, and ZEOFREE® 177 and 265 silica products, all available from Evonik Corporation, and AEROSIL® fumed silicas.

The oral care composition can be substantially free of, essentially free of, or free of a cellulose derivative, such as carboxymethyl cellulose. The oral care composition can comprise less than about 5%, less than about 3%, less than about 2%, less than about 1%, less than about 0.5%, or less than about 0.25%, by weight of the oral care composition, of a cellulose derivative, such as carboxymethyl cellulose. While not wishing to be bound by theory, it is believed that cellulose derivatives, such as carboxymethyl cellulose, can prevent and/or slow the crystallization of oxalic acid within dentin tubules.

The oral care composition can comprise from 0.01% to about 15%, from 0.1% to about 10%, from about 0.2% to about 5%, or from about 0.5% to about 2% of one or more thickening agents.

### Abrasive

The oral care composition of the present invention can comprise an abrasive. Abrasives can be added to oral care formulations to help remove surface stains from teeth. Preferably, the abrasive is a calcium abrasive or a silica abrasive.

The calcium abrasive can be any suitable abrasive compound that can provide calcium ions in an oral care composition and/or deliver calcium ions to the oral cavity when the oral care composition is applied to the oral cavity. The oral care composition can comprise from about 5% to about 70%, from about 10% to about 60%, from about 20% to about 50%, from about 25% to about 40%, or from about 1% to about 50% of a calcium abrasive. The calcium abrasive can comprise one or more calcium abrasive compounds, such as calcium carbonate, precipitated calcium carbonate (PCC), ground calcium carbonate (GCC), chalk, dicalcium phosphate, calcium pyrophosphate, and/or mixtures thereof.

The oral care composition can also comprise a silica abrasive, such as silica gel (by itself, and of any structure), precipitated silica, amorphous precipitated silica (by itself, and of any structure as well), hydrated silica, and/or combinations thereof. The oral care composition can comprise from about 5% to about 70%, from about 10% to about 60%, from about 10% to about 50%, from about 20% to about 50%, from about 25% to about 40%, or from about 1% to about 50% of a silica abrasive.

The oral care composition can also comprise another abrasive, such as bentonite, perlite, titanium dioxide, alumina, hydrated alumina, calcined alumina, aluminum silicate, insoluble sodium metaphosphate, insoluble potassium metaphosphate, insoluble magnesium carbonate, zirconium silicate, particulate thermosetting resins and other suitable abrasive materials. The oral care composition can comprise from about 5% to about 70%, from about 10% to about 60%, from about 10% to about 50%, from about 20% to about 50%, from about 25% to about 40%, or from about 1% to about 50% of another abrasive.

### Amino Acid

The oral care composition can comprise amino acid. The amino acid can comprise one or more amino acids, peptide, and/or polypeptide, as described herein.

Amino acids, as in Formula II, are organic compounds that contain an amine functional group, a carboxyl functional group, and a side chain (R in Formula II) specific to each amino acid. Suitable amino acids include, for example, amino acids with a positive or negative side chain, amino acids with an acidic or basic side chain, amino acids with polar uncharged side chains, amino acids with hydrophobic side chains, and/or combinations thereof. Suitable amino acids also include, for example, arginine, histidine, lysine, aspartic acid, glutamic acid, serine, threonine, asparagine, glutamine, cysteine, selenocysteine, glycine, proline, alanine, valine, isoleucine, leucine, methionine, phenylalanine, tyrosine, tryptophan, citrulline, ornithine, creatine, diaminobutanoic acid, diaminoproprionic acid, salts thereof, and/or combinations thereof.

Suitable amino acids include the compounds described by Formula II, either naturally occurring or synthetically derived. The amino acid can be zwitterionic, neutral, positively charged, or negatively charged based on the R group and the environment. The charge of the amino acid, and whether particular functional groups, can interact with tin at particular pH conditions, would be well known to one of ordinary skill in the art.

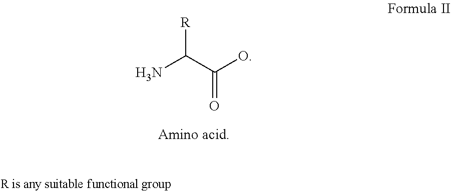

Suitable amino acids include one or more basic amino acids, one or more acidic amino acids, one or more neutral amino acids, or combinations thereof.

The oral care composition can comprise from about 0.01% to about 20%, from about 0.1% to about 10%, from about 0.5% to about 6%, or from about 1% to about 10% of amino acid, by weight of the oral care composition.

The term “neutral amino acids” as used herein include not only naturally occurring neutral amino acids, such as alanine, asparagine, cysteine, glutamine, glycine, isoleucine, leucine, methionine, phenylalanine, proline, serine, threonine, tryptophan, tyrosine, valine, but also biologically acceptable amino acids which have an isoelectric point in range of pH 5.0 to 7.0. The biologically preferred acceptable neutral amino acid has a single amino group and carboxyl group in the molecule or a functional derivative hereof, such as functional derivatives having an altered side chain albeit similar or substantially similar physio chemical properties. In a further embodiment the amino acid would be at minimum partially water soluble and provide a pH of less than 7 in an aqueous solution of 1 g/1000 ml at 25° C.

Accordingly, neutral amino acids suitable for use in the invention include, but are not limited to, alanine, aminobutyrate, asparagine, cysteine, cystine, glutamine, glycine, hydroxyproline, isoleucine, leucine, methionine, phenylalanine, proline, serine, taurine, threonine, tryptophan, tyrosine, valine, salts thereof, or mixtures thereof. Preferably, neutral amino acids used in the composition of the present invention may include asparagine, glutamine, glycine, salts thereof, or mixtures thereof. The neutral amino acids may have an isoelectric point of 5.0, or 5.1, or 5.2, or 5.3, or 5.4, or 5.5, or 5.6, or 5.7, or 5.8, or 5.9, or 6.0, or 6.1, or 6.2, or 6.3, or 6.4, or 6.5, or 6.6, or 6.7, or 6.8, or 6.9, or 7.0, in an aqueous solution at 25° C. Preferably, the neutral amino acid is selected from proline, glutamine, or glycine, more preferably in its free form (i.e. uncomplexed). If the neutral amino acid is in its salt form, suitable salts include salts known in the art to be pharmaceutically acceptable salts considered to be physiologically acceptable in the amounts and concentrations provided.

### Whitening Agent

The oral care composition may comprise from about 0.1% to about 10%, from about 0.2% to about 5%, from about 1% to about 5%, or from about 1% to about 15%, by weight of the oral care composition, of a whitening agent. The whitening agent can be a compound suitable for whitening at least one tooth in the oral cavity. The whitening agent may include peroxides, metal chlorites, perborates, percarbonates, peroxyacids, persulfates, dicarboxylic acids, and combinations thereof. Suitable peroxides include solid peroxides, hydrogen peroxide, urea peroxide, calcium peroxide, benzoyl peroxide, sodium peroxide, barium peroxide, inorganic peroxides, hydroperoxides, organic peroxides, and mixtures thereof. Suitable metal chlorites include calcium chlorite, barium chlorite, magnesium chlorite, lithium chlorite, sodium chlorite, and potassium chlorite. Other suitable whitening agents include sodium persulfate, potassium persulfate, peroxydone, 6-phthalimido peroxy hexanoic acid, pthalamidoperoxycaproic acid, or mixtures thereof.

### Humectant

The oral care composition can comprise one or more humectants, have low levels of a humectant, or be free of a humectant. Humectants serve to add body or “mouth texture” to an oral care composition or dentifrice as well as preventing the dentifrice from drying out. Suitable humectants include polyethylene glycol (at a variety of different molecular weights), propylene glycol, glycerin (glycerol), erythritol, xylitol, sorbitol, mannitol, butylene glycol, lactitol, hydrogenated starch hydrolysates, and/or mixtures thereof. The oral care composition can comprise one or more humectants each at a level of from 0 to about 70%, from about 5% to about 50%, from about 10% to about 60%, or from about 20% to about 80%, by weight of the oral care composition.

### Water

The oral care composition of the present invention can be a dentifrice composition that is anhydrous, a low water formulation, or a high water formulation. In total, the oral care composition can comprise from 0% to about 99%, about 20% or greater, about 30% or greater, about 50% or greater, up to about 45%, or up to about 75%, by weight of the composition, of water. Preferably, the water is USP water.

In a high water dentifrice formulation, the dentifrice composition comprises from about 45% to about 75%, by weight of the composition, of water. The high water dentifrice composition can comprise from about 45% to about 65%, from about 45% to about 55%, or from about 46% to about 54%, by weight of the composition, of water. The water may be added to the high water dentifrice formulation and/or may come into the composition from the inclusion of other ingredients.

In a low water dentifrice formulation, the dentifrice composition comprises from about 10% to about 45%, by weight of the composition, of water. The low water dentifrice composition can comprise from about 10% to about 35%, from about 15% to about 25%, or from about 20% to about 25%, by weight of the composition, of water. The water may be added to the low water dentifrice formulation and/or may come into the composition from the inclusion of other ingredients.

In an anhydrous dentifrice formulation, the dentifrice composition comprises less than about 10%, by weight of the composition, of water. The anhydrous dentifrice composition comprises less than about 5%, less than about 1%, or 0%, by weight of the composition, of water. The water may be added to the anhydrous formulation and/or may come into the dentifrice composition from the inclusion of other ingredients.

The dentifrice composition can also comprise other orally acceptable carrier materials, such as alcohol, humectants, polymers, surfactants, and acceptance improving agents, such as flavoring, sweetening, coloring and/or cooling agents.

The oral care composition can also be a mouth rinse ⁻formulation. A mouth rinse formulation can comprise from about 7.5% to about 99%, from about 75% to about 95%, or from about 80% to about 95% of water.

### Other Ingredients

The oral care composition can comprise a variety of other ingredients, such as flavoring agents, sweeteners, colorants, preservatives, buffering agents, or other ingredients suitable for use in oral care compositions, as described below.

Flavoring agents also can be added to the oral care composition. Suitable flavoring agents include oil of wintergreen, oil of peppermint, oil of spearmint, clove bud oil, menthol, anethole, methyl salicylate, eucalyptol, cassia, 1-menthyl acetate, sage, eugenol, parsley oil, oxanone, alpha-irisone, marjoram, lemon, orange, propenyl guaethol, cinnamon, vanillin, ethyl vanillin, heliotropine, 4-cis-heptenal, diacetyl, methyl-para-tert-butyl phenyl acetate, and mixtures thereof. Coolants may also be part of the flavor system. Preferred coolants in the present compositions are the paramenthan carboxyamide agents such as N-ethyl-p-menthan-3-carboxamide (known commercially as “WS-3”) or N-(Ethoxycarbonylmethyl)-3-p-menthanecarboxamide (known commercially as “WS-5”), and mixtures thereof. A flavor system is generally used in the compositions at levels of from about 0.001% to about 5%, by weight of the oral care composition. These flavoring agents generally comprise mixtures of aldehydes, ketones, esters, phenols, acids, and aliphatic, aromatic and other alcohols.

Sweeteners can be added to the oral care composition to impart a pleasing taste to the product.

Suitable sweeteners include saccharin (as sodium, potassium or calcium saccharin), cyclamate (as a sodium, potassium or calcium salt), acesulfame-K, thaumatin, neohesperidin dihydrochalcone, ammoniated glycyrrhizin, dextrose, levulose, sucrose, mannose, sucralose, stevia, and glucose.

Colorants can be added to improve the aesthetic appearance of the product. Suitable colorants include without limitation those colorants approved by appropriate regulatory bodies such as the FDA and those listed in the European Food and Pharmaceutical Directives and include pigments, such as TiO₂, and colors such as FD&C and D&C dyes.

Preservatives also can be added to the oral care compositions to prevent bacterial growth. Suitable preservatives approved for use in oral compositions such as methylparaben, propylparaben, benzoic acid, and sodium benzoate can be added in safe and effective amounts.

Titanium dioxide may also be added to the present composition. Titanium dioxide is a white powder which adds opacity to the compositions. Titanium dioxide generally comprises from about 0.25% to about 5%, by weight of the oral care composition.

Other ingredients can be used in the oral care composition, such as desensitizing agents, healing agents, other caries preventative agents, chelating/sequestering agents, vitamins, amino acids, proteins, other anti-plaque/anti-calculus agents, opacifiers, antibiotics, anti-enzymes, enzymes, pH control agents, oxidizing agents, antioxidants, and the like.

### Oral Care Composition Forms

Suitable compositions for the delivery of the oxalic acid include emulsion compositions, such as the emulsion compositions of U.S. Patent Application Publication No. 2018/0133121, which is herein incorporated by reference in its entirety, unit-dose compositions, such as the unit-dose compositions of U.S. Patent Application Publication No. 2019/0343732, which is herein incorporated by reference in its entirety, leave-on oral care compositions, such as the leave-on oral care compositions of U.S. patent application Ser. No. 16/899,834, which is herein incorporated by reference in its entirety, jammed emulsions, such as the jammed oil-in-water emulsion compositions of U.S. Pat. No. 10,780,032, which is herein incorporated by reference in its entirety, dentifrice compositions, mouth rinse compositions, mouthwash compositions, tooth gel, subgingival gel, mouth rinse, mousse, foam, mouth spray, lozenge, chewable tablet, chewing gum, tooth whitening strips, floss and floss coatings, breath freshening dissolvable strips, denture care products, denture adhesive products, or combinations thereof.

### Oral Care Regimen

The oxalic acid can be delivered in the same composition as tin and/or fluoride or the oxalic acid can be delivered in a separate composition. For example, a first composition can comprise tin and/or fluoride and a second composition can comprise oxalic acid and pH buffering agent. The first and second composition can be delivered simultaneously, such as in a dual-phase composition or sequentially from discrete compositions.

An oral care kit can include the first composition comprising tin and/or fluoride and the second composition comprising oxalic acid and pH buffering agent. The oral care kit can also include instructions directing a user to apply the first composition to an oral cavity of the user followed by applying the second composition to the oral cavity of the user. The first composition can be expectorated prior to the application of the second composition or the second composition can be applied prior to the expectoration of the first composition from the oral cavity.

The entire oral care regimen can have a duration of from one minute to about three minutes with each application step having a duration of from about 30 seconds to about 2 minutes or about 1 minute.

The components can be delivered to the oral cavity simultaneously or sequentially. The simplest case is simultaneous, continuous delivery of equal amounts of the two components or a constant ratio of the components during a single oral care session. The two components may be provided separately, such as in a dual-phase composition in two separate compositions, and then delivered simultaneously to the oral cavity. Brushing duration is sufficiently short so that the components will not be inactivated. Another use for simultaneous, continuous delivery is systems that include two components that react relatively slowly, and that will remain in the oral cavity after brushing to be absorbed by the teeth and or gums.

In the case of sequential delivery, both components may be delivered during a single oral care session, e.g., a single brushing session or other single treatment session (single use, start to finish, by a particular user, typically about 0.1 to 5 minutes), or alternatively the components may be delivered individually over multiple oral care sessions. Many combinations are possible, for example delivery of both components during a first oral care session and delivery of only one of the components during a second oral care session.

Sequential delivery during a single oral care session may take various forms. In one case, two components are delivered in alternation, as either a few relatively long duration cycles during brushing (A B A B), or many rapid-fire alternations (A BABABABAB . . . A B).

In another case, two or more components are delivered one after the other during a single oral care session, with no subsequent alternating delivery in that oral care session (A followed by B). For example, a first composition comprising fluoride and/or tin can be delivered initially, to initiate brushing and provide cleansing, followed by a second composition comprising oxalic acid.

The first composition comprising tin and/or fluoride and the second composition comprising oxalic acid and pH buffering agent can be alternated at different oral care sessions. For example, the user can be directed to use first composition at one point in a day, such as in the morning, and to use the second composition at second point in the day, such as in the afternoon or evening.

### EXAMPLES

The invention is further illustrated by the following examples, which are not to be construed in any way as imposing limitations to the scope of this invention. Various other aspects, modifications, and equivalents thereof which, after reading the description herein, may suggest themselves to one of ordinary skill in the art without departing from the spirit of the present invention or the scope of the appended claims.

TABLE 1
Example Compositions

| | Ex. 1 | Ex. 2 | Ex. 3 | Ex. 4 | Ex. 5 | Ex. 6 |
|---|---|---|---|---|---|---|
| | (wt %) | (wt %) | (wt %) | (wt %)% | (wt %)% | (wt %) |
| Sorbitol | 62.000000 | 64.000000 | 64.000000 | 64.000000 | 64.000000 | 45.0000 |
| Water | 8.1450 | 8.5850 | 7.3700 | 6.2170 | 7.2700 | 19.1091 |
| SnF₂ | — | — | — | — | — | 0.4540 |
| SnCl₂/10% silica blend | — | — | — | — | — | 0.5619 |
| NaF | — | — | — | 0.2430 | — | — |
| Sodium | — | — | 1.1400 | — | 1.1400 | — |
| Monofluorophosphate | | | | | | |
| Sodium Gluconate | — | — | — | 1.0000 | — | 1.3000 |
| NaOH 50% | — | — | — | — | — | 0.1500 |
| Saccharin | 0.4000 | 0.4000 | 0.4000 | 0.4000 | 0.4000 | 0.3500 |
| Sucralose Solution (25%) | — | — | — | — | — | 0.0800 |
| Xanthan Gum | 0.5000 | 0.5000 | 0.5000 | 0.5000 | 0.6000 | 0.8750 |
| Carboxymethylcellulose | 1.0000 | 1.0000 | 1.0000 | 1.0000 | — | — |
| Carrageenan | — | — | — | — | 1.0000 | 1.5000 |
| Citric Acid | 0.2150 | 0.2750 | 0.3500 | 0.7000 | 0.3500 | — |
| Na Citrate | — | — | — | 0.7000 | — | 1.2050 |
| Potassium oxalate | 3.1400 | 3.1400 | 3.1400 | 3.1400 | 3.1400 | 3.1400 |
| monohydrate | | | | | | |
| TiO₂ | 0.5000 | 0.5000 | 0.5000 | 0.5000 | 0.5000 | 0.5000 |
| Thickening Silica (Z165) | 3.0000 | 0.5000 | 0.5000 | 0.5000 | 0.5000 | — |
| Abrasive Silica (Z119) | 15.0000 | 15.0000 | 15.0000 | 15.0000 | 15.0000 | 17.5000 |
| SLS Solution (28%) | 5.0000 | 5.0000 | 5.0000 | 5.0000 | 5.0000 | 7.0000 |
| Flavor | 1.1000 | 1.1000 | 1.1000 | 1.1000 | 1.1000 | 1.2750 |

TABLE 1 includes various example oral care compositions. Ex. 1 included potassium oxalate monohydrate and citric acid (pH buffering agent). Ex. 2 included potassium oxalate monohydrate and citric acid (pH buffering agent). Ex. 3 included potassium oxalate monohydrate, citric acid (pH buffering agent), and sodium monofluorophosphate. Ex. 4 included potassium oxalate monohydrate, citric acid (pH buffering agent), sodium citrate (pH buffering agent), and sodium fluoride. Ex. 5 included potassium oxalate monohydrate, citric acid (pH buffering agent), and sodium monofluorophosphate. Ex. 6 potassium oxalate monohydrate, sodium citrate (pH buffering agent), and stannous fluoride.

### Models

Two independent models were utilized to experimentally evaluate the performance of test products. First, an in vitro hydrodynamic flow model (HF model) was utilized to evaluate volumetric flow through coronal human dentin sections before and after application of test solutions. Second, an in situ salivary dilution and neutralization model (SDN model) was utilized to understand the effect of the oral environment on the concentration and pH of oxalate in each treatment during application.

### In Vitro Hydrodynamic Flow Cell Performance Model (HF Model)

Performance of antisensitivity solutions was assessed via quantitative measurement of volumetric liquid flow, also commonly known as hydrodynamic flow, through coronal human dentin. Dentin samples were prepared by sectioning maxillary and mandibular third molars parallel to the occlusal surface to obtain a disk approximately 0.8 mm thick. Disks were then etched in a 40 khz sonic bath for 2 minutes per side in 6% citric acid solution (pH 1.8), rinsed in distilled water, and stored in a small aliquot of a lactated Ringers solution (at 5° C.) until use.

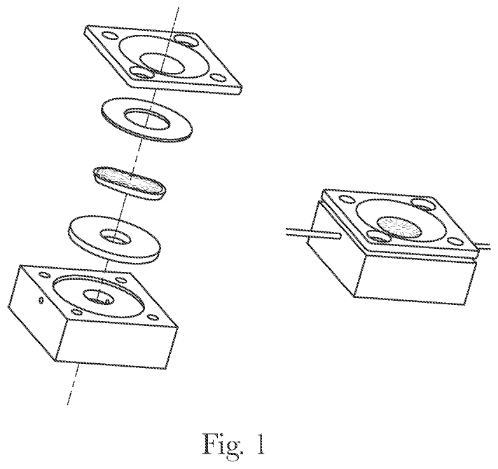

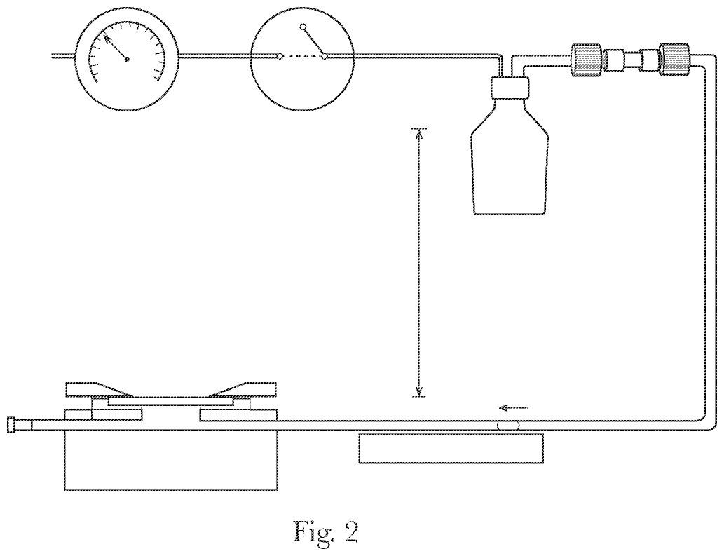

Dentin samples were mounted in the split cell shown in FIG. 1 and FIG. 2, which permitted application of fluid pressure from the cervical side of the dentin section while providing access to the coronal surface for treatment without disassembly of the cell. The liquid utilized in the flow model is a lactated Ringers solution (buffer), with an adjusted pH of 7.0. In order to obtain a stable background flow rate, the dentin section was conditioned via application of a power toothbrush (Oral-B Professional Care SmartSeries 5000) for several minutes while the section was subjected to 5 psi (34.5 kPa) of liquid flow of buffer from the cervical side. Volumetric flow through the dentin was then quantified with a digital flowmeter (CorSolutions p/n FM-Micro-1X). Flow stability was confirmed by three successive baseline flow measurements, each separated by a mechanical challenge consisting of sonic brushing the coronal surface for 30 seconds using the power toothbrush with 100 g of force. If the three flow measurements varied by less than 10%, a baseline value was calculated from the average. Specimens which exhibited unstable flow after multiple conditioning cycles were discarded.

The experimental protocol consisted of (1) reducing the applied liquid pressure to that exerted by a simple 15 cm head, (2) applying 200 μL of treatment solution/saliva mixture to the surface of the conditioned dentin sample, brushing with powerbrush in sensi mode for 60 seconds, (3) thorough rinsing of the dentin surface with buffer, and (4) a single observation of post-treatment flow under 5 psi of liquid pressure. Results were expressed as % reduction in volumetric flow or hydraulic conductance as per Equation 1, below.

$$Calculation of Flow Reduction \\ \% Flow Reduction = 100 \frac{( {Q}_{p} - {Q}_{b} )}{{Q}_{b}} \\ Where \\ {Q}_{p} = post \text{-} treatment flow or hydraulic conductance , \\ and \\ {Q}_{b} = average baseline flow or hydraulic conductance \qquad EQUATION 1$$

### In Situ Salivary Dilution and Neutralization Model (SDN Model)

The in situ SDN model involved collection and analysis of a baseline saliva sample and several expectorant samples from a volunteer at regular intervals post-treatment. Sample analysis involved collection of all expectorant weights and determination of neat pH by electrode.

### Results

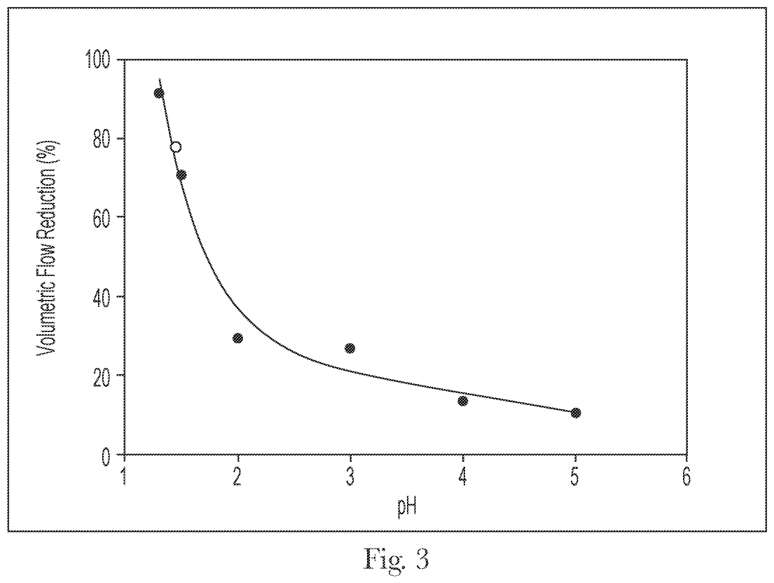

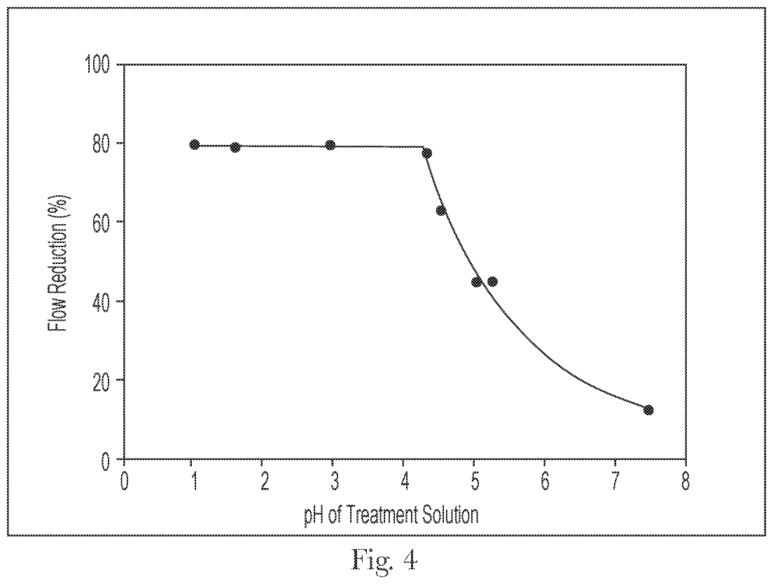

FIGS. 3 and 4 show flow reduction in human dentin in the HF model following exposure to oxalate vs. treatment pH. An aqueous solution, in which oxalate concentration was held constant and in which pH was varied, served as a simplified product matrix to illustrate that oxalate performance is a strong and inverse function of pH.

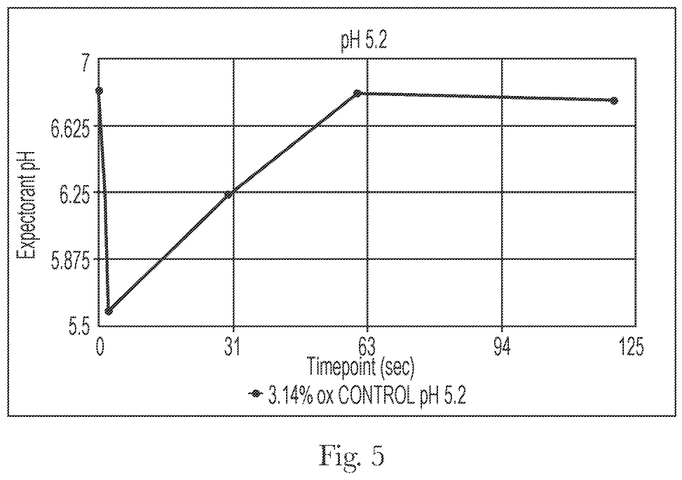

FIG. 5 illustrates the behavior of a moderately acidic oral care product when introduced to the oral cavity as observed with the SDN model. While application of this simplified product was found to initially reduce the pH of oral fluids, the pH drop was surprisingly transient. Specifically, the pH of oral fluids essentially reverted back to baseline values within the time period associated with a typical product application (i.e. 1-2 minutes). This experiment highlights the difficulty of maintaining a constant environment in the oral cavity for optimal product performance when key ingredients are in contact with oral tissues for a finite period of time.

Investigation of buffering to maintain oral pH: As a result of the observation of the rapid rebound in pH of expectorated saliva, the effect of buffering treatment solutions was studied. Three control solutions containing 3.14% potassium oxalate monohydrate (K2ox) (1.5% oxalate ion) were adjusted to pH 4.2, pH 5.2 and pH 7.0. Treatment solutions also containing 3.14% K2ox were buffered at pH 4.2, 5.2, and 7.0 with 0.2 M citric, malonic, and carbonic acid buffers, respectively. In each case NaOH or HCl were used to adjust buffered solutions to the desired pH endpoint. Baseline saliva samples were collected before treatment application (time=0), while post-treatment expectorant samples were collected at the following time intervals; immediate (2 seconds), 30 seconds, 60 seconds and 120 seconds following product introduction into the oral cavity. Usage instructions for the treatment solutions required swishing of 1.0 mL for the specified time interval, contacting all oral soft tissue surfaces.

Final saliva weight and pH were measured for each sample. Measurement of pH was performed on undiluted samples using a Cole Parmer flat-tip pH electrode p/n 05990-65. Stimulated saliva weight data and pH data were collected and are shown in Table 1 at the 30 second treatment interval, which may be considered as the half-way point during a typical 60 second product application window.

Measured pH values of expectorated saliva 30 s post-treatment, when treatment solutions were buffered at pH 4.2, 5.2, and 7.0 using 0.2 M citric, malonic, and carbonic acid buffers, respectively, are shown in Table 1.

TABLE 1
in situ Saliva: Treatment Solution Ratios and Measured pH

| | | | | | Theoretical | |
|---|---|---|---|---|---|---|
| | | In situ | Total weight (g) | | K2Ox (wt %) | in situ |
| | Treatment | Treatment | of expectorated | Expectorated | after salivary | measured |
| Formulated pH | Description | volume (mL) | fluids at 30 sec | saliva (g) | dilution | pH |
| 4.2 | Control | 1.0 | 2.6 | 1.6 | 1.2 | 4.73 |
| | Buffered | 1.0 | 3.3 | 2.3 | 0.95 | 4.42 |
| 5.2 | Control | 1.0 | 1.85 | 0.85 | 1.7 | 6.47 |
| | Buffered | 1.0 | 2.7 | 1.7 | 1.2 | 5.33 |
| 7.0 | Control | 1.0 | 1.6 | 0.60 | 2.0 | 6.76 |
| | Buffered | 1.0 | 2.1 | 1.1 | 1.5 | 7.76 |

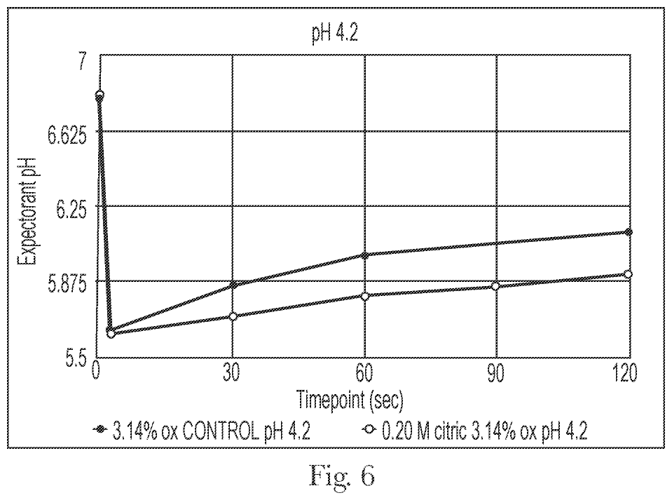

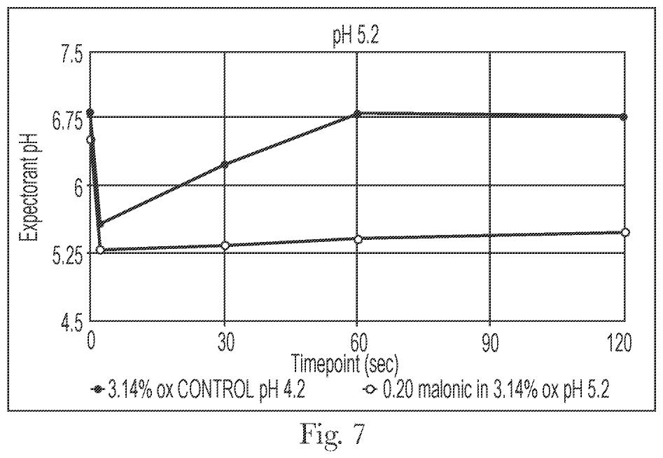

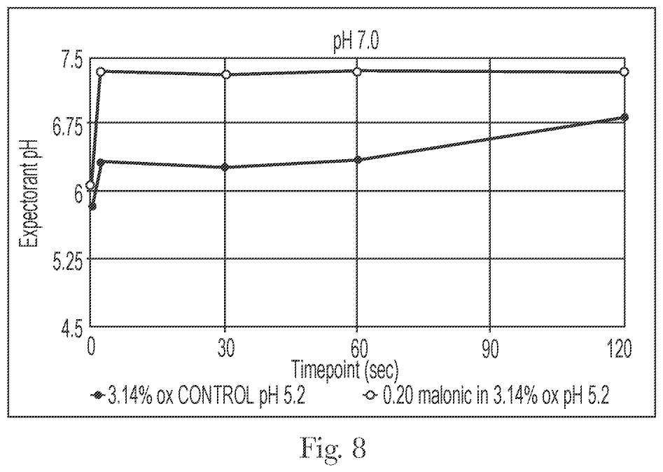

The data in Table 1 clearly shows the ability of buffered solutions to maintain in situ pH approximately at the formulated pH, whereas unbuffered control solutions rebound almost immediately toward baseline pH as shown in FIGS. 6-8. It is clear, that addition of a buffer is most impactful in the pH range of about 5.2. While not wishing to being bound by theory, it is believed that this phenomenon is in part explained by the fact that oxalate ion itself functions as a reasonably strong buffer in the pH 4.2 solutions, having a pKa of 4.1. Importantly, it was observed that the addition of buffering agents to the K2ox treatment solution caused increased salivary stimulation, which resulted in additional dilution of oxalate ion in situ, as shown in Table 1 above.

### Investigation of Buffering and Salivary Dilution on HF Performance:

While the SDN model showed that addition of buffering agents to treatment solutions more effectively maintained desired oral pH during product application, the associated increase in salivary dilution made unclear the overall effect of buffering on antisensitivity product performance. To determine if buffering improved the ability of acidic treatment solutions to actually reduce pulpal flow despite increased oxalate dilution, control and treatment solutions were evaluated in the HF model. In this case, control and treatment solutions consisted of two-part mixtures of potassium oxalate solutions and whole, pooled, stimulated human saliva, where dilution ratios matched the in situ dilution measurements summarized in Table 1. The actual treatment ratios are given in Table 2, below. After mixing saliva and treatment solutions, a 200 μL aliquot of the mixture was applied to the surface of the dentin. The “in vitro treatment pH” is the measured value of the diluted treatment after mixing, again matching measured in situ SDN data at 30 seconds.

TABLE 2
Oxalate Solution: Saliva Treatment Ratios Applied in vitro

| | | | | In vitro | |
|---|---|---|---|---|---|
| | | | In vitro | oxalate | |
| | | | saliva | solution | |
| | | Treatment | volume | volume | In vitro |
| Example | Formulated pH | Description | (uL) | (uL) | treatment pH |
| 7 | 4.2 | Control | 160 | 100 | 4.48 |
| 8 | | Buffered | 230 | 100 | 4.32 |
| 9 | 5.2 | Control | 85 | 100 | 6.57 |
| 10 | | Buffered | 180 | 100 | 5.51 |
| 11 | 7.0 | Control | 60 | 100 | 7.44 |
| 12 | | Buffered | 110 | 100 | 7.41 |

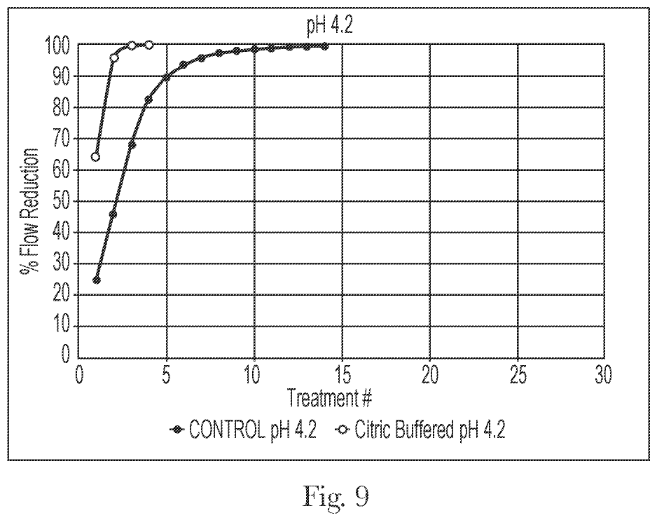

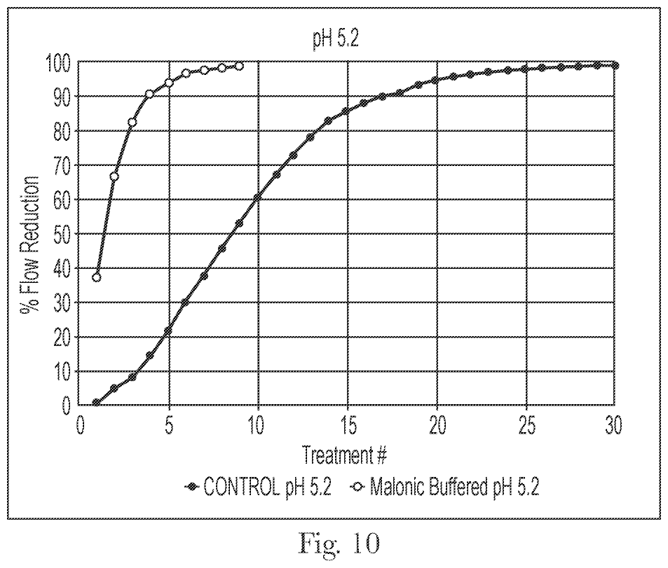

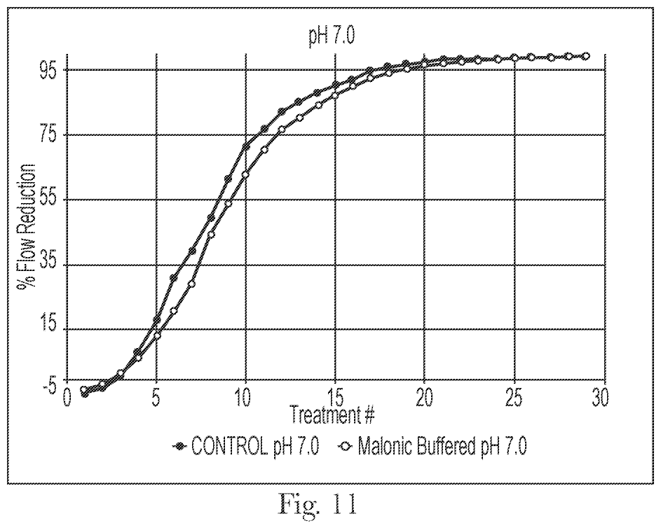

One measure of treatment solution efficacy is how quickly the dentin is occluded, i.e. the number of product application cycles required to effect occlusion. FIGS. 9-11 show reduction in hydrodynamic flow as a function of application cycles. Table 3 summarizes the number of application cycles required to reach full occlusion. In this case, full occlusion is defined as approximately 99% flow reduction from baseline value.

TABLE 3
Treatment Efficacy in vitro

| | | | Number of |
|---|---|---|---|
| | | | Treatment |
| | | | Applications to |
| | | Treatment | Reach Full |
| Example | Formulated pH | Description | Occlusion |
| 7 | 4.2 | Control | 11 |
| 8 | | Buffered | 3 |
| 9 | 5.2 | Control | 30 |
| 10 | | Buffered | 9 |
| 11 | 7.0 | Control | 26 |
| 12 | | Buffered | 28 |

Interestingly, the HF model shows that overall oxalate performance is significantly enhanced at pH 5.2 and 4.2 by the addition of buffering agents despite the dilution effects caused by increased stimulation of saliva. No significant enhancement was observed from buffering in the pH 7 region. It should be noted that the performance benefit resulting from formulation at low pH must be balanced against the increasing potential for damage to hard tissues, i.e. acid erosion of dentin and enamel. The region near pH 5.2 may be of particular interest, especially when formulating abrasive products such as dentifrice, as abrasion of acid-softened enamel is of special and increasingly greater concern at lower pH values.

Empirical evaluation of product performance using the HF model predicts a material performance benefit for oxalate-based antisensitivity products when the acidity of oral fluids is maintained during product application. The SDN model shows that while addition of a buffering agent successfully maintains moderate acidity during product application, these buffers also cause salivary stimulation, resulting in substantial dilution of the oxalate ion in situ. Even when salivary dilution is appropriately accounted for in the HF model, overall performance is substantially improved by the presence of a buffer, particularly in the region near pH 5.2.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention

1. An oral care composition comprising:
(a) oxalic acid; and
(b) pH buffering agent with a pKa of from about 4 to about 6.5.

2. The oral care composition of claim 1, wherein the pH buffering agent comprises monocarboxylic acid, dicarboxylic acid, tricarboxylic acid, tetracarboxylic acid, or combinations thereof.

3. The oral care composition of claim 2, wherein the pH buffering agent comprises monocarboxylic acid.

4. The oral care composition of claim 1, wherein the pH buffering agent comprises adipic acid, glutaric acid, succinic acid, malonic acid, glutamic acid, ascorbic acid, citric acid, salts thereof, or combinations thereof.

5. The oral care composition of claim L wherein the oxalic acid comprises oxalic acid, monosodium monohydrogen oxalate, disodium oxalate, monopotassium monohydrogen oxalate, dipotassium oxalate, calcium oxalate, or combinations thereof.

6. The oral care composition of claim 1, wherein the pH buffering agent has a pKₐ of from about 4.5 to about 6.

7. The oral care composition of claim 1, wherein the oral care composition is configured to maintain a pH of from about 4 to about 6 in an oral cavity of a user.

8. The oral care composition of claim 1, wherein the oral care composition comprises fluoride.

9. The oral care composition of claim 8, wherein the fluoride comprises sodium fluoride, sodium monofluorophosphate, amine fluoride, stannous fluoride, or combinations thereof.

10. The oral care composition of claim 1, wherein the oral care composition comprising metal.

11. The oral care composition of claim 10, wherein the metal comprises tin, zinc, copper, or combinations thereof.

12. The oral care composition of claim 11, wherein the tin comprises stannous fluoride, stannous chloride, or combinations thereof.

13. The oral care composition of claim 11, wherein the zinc comprises zinc citrate, zinc oxide, zinc phosphate, zinc lactate, or combinations thereof.

14. The oral care composition of claim 1, wherein the oral care composition is free of, essentially free of, or substantially free of zinc.

15. The oral care composition of claim 1, wherein the oral care composition comprises thickening agent.

16. The oral care composition of claim 15, wherein the thickening agent comprises polysaccharide, polymer, silica thickener, or combinations thereof.

17. The oral care composition of claim 15, wherein the thickening agent is free of, essentially free of, or substantially free of cellulose, carboxymethyl cellulose, or combinations thereof.

18. The oral care composition of claim 1, wherein the oral care composition comprises polyphosphate.

19. The oral care composition of claim 18, wherein the polyphosphate comprises pyrophosphate, tripolyphosphate, tetrapolyphosphate, hexametaphosphate, or combinations thereof.

20. The oral care composition of claim 1, wherein the oral care composition is free of, essentially free of, or substantially free of polyphosphate.

21. The oral care composition of claim 1, wherein the oral care composition is free of, essentially free of, or substantially free of orthophosphate.

22. The oral care composition of claim 1, wherein the oral care composition comprises abrasive.

23. The oral care composition of claim 22, wherein the abrasive comprises silica abrasive, calcium abrasive, or combinations thereof.

24. The oral care composition of claim 23, wherein the silica abrasive comprises precipitated silica.

25. The oral care composition of claim 23, wherein the calcium abrasive comprises calcium carbonate, calcium pyrophosphate, calcium phosphate, hydroxyapatite, or combinations thereof.

26. The oral care composition of claim 1, wherein the oral care composition has a pH of from about 4 to about 6.5.

27. The oral care composition of claim 26, wherein the pH is from about 4 to about 6.

28. The oral care composition of claim 26, wherein the pH is from about 4 to about 5.5.

29. A method of preventing, treating, or mitigating sensitivity in an oral cavity of a user, the method comprising:
(a) directing the user to apply the oral care composition of claim 1 to at least one tooth in the oral cavity for an application period of from about 30 seconds to about 2 minutes: and
(b) directing the user to expectorate the oral care composition after the application period.

30. The method of claim 29, wherein the oral care composition does not form a film, varnish, or combinations thereof in the oral cavity.
